(19) United States
(12) Patent Application Publication
(10) Pub. No.: US 2021/0065882 A1
(43) Pub. Date: Mar. 4, 2021

(54) METHOD AND SYSTEM FOR PROMPTING DATA DONATION FOR ARTIFICIAL INTELLIGENCE TOOL DEVELOPMENT

(71) Applicant: GE Precision Healthcare LLC, Wauwatosa, WI (US)

(72) Inventor: Kristin Sarah McLeod, Oslo (NO)

(21) Appl. No.: 16/559,269

(22) Filed: Sep. 3, 2019

(51) Int. Cl. G16H 30/40; G06T 7/187; G06F 3/0482; G06F 9/54

(52) U.S. Cl. CPC G16H 30/40; G06T 7/187; G06T 2207/10132; G06F 9/542; G06F 3/0482

(57) ABSTRACT

Systems and methods for prompting data donation for artificial intelligence tool development are provided. The method includes presenting an ultrasound image and at least one automated analysis feature at a display system. The at least one automated analysis feature includes one or more non-enabled automated analysis features. The method includes receiving a user selection of at least one of the one or more non-enabled automated analysis features. The method includes presenting at the display system a prompt providing a user option to share user analysis data. The method includes receiving a user selection opting to share the user analysis data. The method includes providing access to the at least one of the one or more non-enabled automated analysis features when a condition is met.

### FIELD

Certain embodiments relate to ultrasound imaging. More specifically, certain embodiments relate to a method and system providing artificial intelligence tool development by prompting user data donation.

### BACKGROUND

Ultrasound imaging is a medical imaging technique for imaging organs and soft tissues in a human body. Ultrasound imaging uses real time, non-invasive high frequency sound waves to produce a series of two-dimensional (2D) and/or three-dimensional (3D) images.

Artificial intelligence processing of ultrasound images and/or video is often applied to process the images and/or video to assist an ultrasound operator or other medical personnel viewing the processed image data with providing a diagnosis. For example, artificial intelligence tools may be applied to ultrasound images to automatically provide annotations, measurements, and/or diagnosis that may be presented with the ultrasound images. However, artificial intelligence algorithms are typically developed using thousands of images that have been manually analyzed and provided with annotations, measurements, and/or diagnosis. The accuracy of the artificial intelligence depends in part on the amount of samples used to develop the algorithm, the quality of the samples, the quality of the analysis accompanying the samples, the demographic diversity of the samples, and the like.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A system and/or method is provided for prompting data donation for artificial intelligence tool development, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to prompt data donation for artificial intelligence tool development, in accordance with various embodiments.

FIG. 2 is a block diagram of an exemplary medical workstation that is operable to prompt data donation for artificial intelligence tool development, in accordance with various embodiments.

FIG. 3 is a block diagram of an exemplary system in which a representative embodiment may be practiced.

FIG. 4 is a display of an exemplary ultrasound image and tools for analyzing the ultrasound image, in accordance with various embodiments.

FIG. 5 is a display of an exemplary ultrasound image, tools for analyzing the ultrasound image, and a prompt for donating data, in accordance with various embodiments.

FIG. 6 is a flow chart illustrating exemplary steps that may be utilized for prompting data donation for artificial intelligence tool development, in accordance with various embodiments.

### DETAILED DESCRIPTION

Certain embodiments may be found in a method and system for prompting data donation for artificial intelligence tool development. Various embodiments have the technical effect of providing access to non-enabled automated analysis features in exchange for sharing user analysis data. Aspects of the present disclosure have the technical effect of facilitating donation of user analysis data for the development of artificial intelligence tools.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode (2D mode), M-mode, three-dimensional (3D) mode, CF-mode, PW Doppler, CW Doppler, MGD, and/or sub-modes of B-mode and/or CF such as Shear Wave Elasticity Imaging (SWEI), TVI, Angio, B-flow, BMI, BMI_Angio, and in some cases also MM, CM, TVD where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

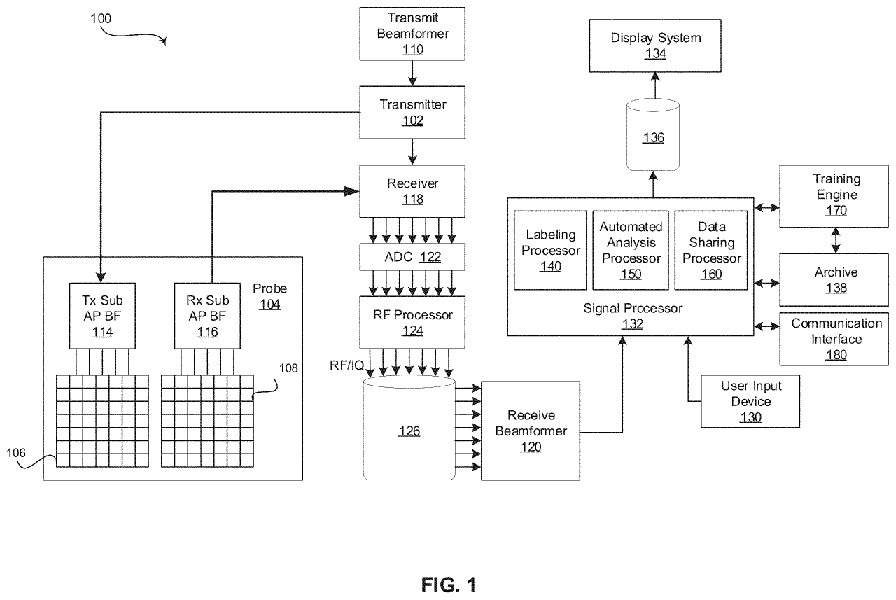

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to prompt data donation for artificial intelligence tool development, in accordance with various embodiments. Referring to FIG. 1, there is shown an ultrasound system 100. The ultrasound system 100 comprises a transmitter 102, an ultrasound probe 104, a transmit beamformer 110, a receiver 118, a receive beamformer 120, A/D converters 122, a RF processor 124, a RF/IQ buffer 126, a user input device 130, a signal processor 132, an image buffer 136, a display system 134, an archive 138, a training engine 170, and a communication interface 180.

The transmitter 102 may comprise suitable logic, circuitry, interfaces and/or code that may be operable to drive an ultrasound probe 104. The ultrasound probe 104 may comprise a two dimensional (2D) array of piezoelectric elements. The ultrasound probe 104 may comprise a group of transmit transducer elements 106 and a group of receive transducer elements 108, that normally constitute the same elements. In certain embodiment, the ultrasound probe 104 may be operable to acquire ultrasound image data covering at least a substantial portion of an anatomy, such as the heart, a blood vessel, or any suitable anatomical structure.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit ultrasonic transmit signals into a region of interest (e.g., human, animal, underground cavity, physical structure and the like). The transmitted ultrasonic signals may be back-scattered from structures in the object of interest, like blood cells or tissue, to produce echoes. The echoes are received by the receive transducer elements 108.

The group of receive transducer elements 108 in the ultrasound probe 104 may be operable to convert the received echoes into analog signals, undergo sub-aperture beamforming by a receive sub-aperture beamformer 116 and are then communicated to a receiver 118. The receiver 118 may comprise suitable logic, circuitry, interfaces and/or code that may be operable to receive the signals from the receive sub-aperture beamformer 116. The analog signals may be communicated to one or more of the plurality of A/D converters 122.

The plurality of A/D converters 122 may comprise suitable logic, circuitry, interfaces and/or code that may be operable to convert the analog signals from the receiver 118 to corresponding digital signals. The plurality of A/D converters 122 are disposed between the receiver 118 and the RF processor 124. Notwithstanding, the disclosure is not limited in this regard. Accordingly, in some embodiments, the plurality of A/D converters 122 may be integrated within the receiver 118.

The RF processor 124 may comprise suitable logic, circuitry, interfaces and/or code that may be operable to demodulate the digital signals output by the plurality of A/D converters 122. In accordance with an embodiment, the RF processor 124 may comprise a complex demodulator (not shown) that is operable to demodulate the digital signals to form I/Q data pairs that are representative of the corresponding echo signals. The RF or I/Q signal data may then be communicated to an RF/IQ buffer 126. The RF/IQ buffer 126 may comprise suitable logic, circuitry, interfaces and/or code that may be operable to provide temporary storage of the RF or I/Q signal data, which is generated by the RF processor 124.

The receive beamformer 120 may comprise suitable logic, circuitry, interfaces and/or code that may be operable to perform digital beamforming processing to, for example, sum the delayed channel signals received from RF processor 124 via the RF/IQ buffer 126 and output a beam summed signal. The resulting processed information may be the beam summed signal that is output from the receive beamformer 120 and communicated to the signal processor 132. In accordance with some embodiments, the receiver 118, the plurality of A/D converters 122, the RF processor 124, and the beamformer 120 may be integrated into a single beamformer, which may be digital. In various embodiments, the ultrasound system 100 comprises a plurality of receive beamformers 120.

The user input device 130 may be utilized to input patient data, scan parameters, settings, select protocols and/or templates, annotate displayed images, perform measurements on displayed images, select automated analysis features and/or tools, and the like. In an exemplary embodiment, the user input device 130 may be operable to configure, manage and/or control operation of one or more components and/or modules in the ultrasound system 100. In this regard, the user input device 130 may be operable to configure, manage and/or control operation of the transmitter 102, the ultrasound probe 104, the transmit beamformer 110, the receiver 118, the receive beamformer 120, the RF processor 124, the RF/IQ buffer 126, the user input device 130, the signal processor 132, the image buffer 136, the display system 134, the archive 138, the training engine 170, and/or the communication interface 180. The user input device 130 may include button(s), rotary encoder(s), a touchscreen, motion tracking, voice recognition, a mousing device, keyboard, camera and/or any other device capable of receiving a user directive. In certain embodiments, one or more of the user input devices 130 may be integrated into other components, such as the display system 134, for example. As an example, user input device 130 may include a touchscreen display.

In various embodiments, anatomical structure depicted in image data may be labeled and/or measured in response to a directive received via the user input module 130. In certain embodiments, automated analysis features and/or tools may be selected in response to a feature selection directive received via the user input module 130. In a representative embodiment, user analysis data may be shared in response to a data donation directive received via the user input module 130.

The signal processor 132 may comprise suitable logic, circuitry, interfaces and/or code that may be operable to process ultrasound scan data (i.e., summed IQ signal) for generating ultrasound images for presentation on a display system 134. The signal processor 132 is operable to perform one or more processing operations according to a plurality of selectable ultrasound modalities on the acquired ultrasound scan data. In an exemplary embodiment, the signal processor 132 may be operable to perform display processing and/or control processing, among other things. Acquired ultrasound scan data may be processed in real-time during a scanning session as the echo signals are received. Additionally or alternatively, the ultrasound scan data may be stored temporarily in the RF/IQ buffer 126 during a scanning session and processed in less than real-time in a live or off-line operation. In various embodiments, the processed image data can be presented at the display system 134 and/or may be stored at the archive 138. The archive 138 may be a local archive, a Picture Archiving and Communication System (PACS), or any suitable device for storing images and related information.

The signal processor 132 may be one or more central processing units, microprocessors, microcontrollers, and/or the like. The signal processor 132 may be an integrated component, or may be distributed across various locations, for example. In an exemplary embodiment, the signal processor 132 may comprise a labeling processor 140, an automated analysis processor 150, and a data sharing processor 160. The signal processor 132 may be capable of receiving input information from a user input device 130 and/or archive 138, generating an output displayable by a display system 134, and manipulating the output in response to input information from a user input device 130, among other things. The signal processor 132, including the labeling processor 140, automated analysis processor 150, and data sharing processor 160, may be capable of executing any of the method(s) and/or set(s) of instructions discussed herein in accordance with the various embodiments, for example.

The ultrasound system 100 may be operable to continuously acquire ultrasound scan data at a frame rate that is suitable for the imaging situation in question. Typical frame rates range from 20-120 but may be lower or higher. The acquired ultrasound scan data may be displayed on the display system 134 at a display-rate that can be the same as the frame rate, or slower or faster. An image buffer 136 is included for storing processed frames of acquired ultrasound scan data that are not scheduled to be displayed immediately. Preferably, the image buffer 136 is of sufficient capacity to store at least several minutes' worth of frames of ultrasound scan data. The frames of ultrasound scan data are stored in a manner to facilitate retrieval thereof according to its order or time of acquisition. The image buffer 136 may be embodied as any known data storage medium.

The signal processor 132 may include a labeling processor 140 that comprises suitable logic, circuitry, interfaces and/or code that may be operable to label, for example, biological and/or artificial structures in ultrasound images presented at the display system 134 with annotations, measurements, diagnosis, and the like in response to user directives provided via the user input device 130. The structures may include artificial structures, such as a needle, catheter, or the like. The structures may include anatomical structures, such as structures of the heart, lungs, fetus, or any suitable internal body structures. For example, with reference to a heart, a user may provide directives via the user input device 130 to the labeling processor 140 for labeling a mitral valve, aortic valve, ventricle chambers, atria chambers, septum, papillary muscle, inferior wall, and/or any suitable heart structure. As another example, a user may provide directives via the user input device 130 to labeling processor 140 for performing heart measurements, such as a left ventricle internal diameter at end systole (LVIDs) measurement, an interventricular septum at end systole (IVSs) measurement, a left ventricle posterior wall at end systole (LVPWs) measurement, or an aortic valve diameter (AV Diam) measurement, among other things. The user may provide directives via the user input device 130 to labeling processor 140 for associating a diagnosis with an ultrasound image. For example, the user may select a diagnosis from a drop down menu, enter text overlaid on the ultrasound image, and/or direct the labeling processor 140 to retrieve a diagnosis from a report. The labeling processor 140 may superimpose the annotations, measurements, diagnosis, and the like provided via the user input device 130 on the ultrasound image presented at the display system 134 or otherwise associate the annotations, measurements, diagnosis, and the like with the ultrasound image. For example, each of the annotations, measurements, and/or diagnosis associated with the ultrasound images may be stored with or in relation to the associated ultrasound image as metadata. In various embodiments, the metadata may include a set of coordinates corresponding with the location of the annotation, measurement, and/or diagnosis in the ultrasound image. The annotations, measurements, and/or diagnosis having the set of coordinates may be stored at archive 138 and/or at any suitable storage medium.

The signal processor 132 may include an automated analysis processor 150 that comprises suitable logic, circuitry, interfaces and/or code that may be operable to apply automated analysis features and/or tools that automatically analyze ultrasound images to identify, segment, annotate, perform measurements, provide diagnosis, and/or the like to structures depicted in the ultrasound images. The biological structures may include, for example, nerves, vessels, organ, tissue, or any suitable biological structures. The artificial structures may include, for example, a needle, an implantable device, or any suitable artificial structures. The automated analysis processor 150 may include artificial intelligence image analysis algorithms, one or more deep neural networks (e.g., a convolutional neural network) and/or may utilize any suitable form of artificial intelligence image analysis techniques or machine learning processing functionality configured to provide the automated analysis feature(s) and/or tool(s).

The automated analysis processor 150 may comprise suitable logic, circuitry, interfaces and/or code that may be operable to annotate, perform measurements, and/or provide diagnosis to structures depicted in ultrasound images. In various embodiments, the automated analysis processor 150 may be provided as a deep neural network that may be made up of, for example, an input layer, an output layer, and one or more hidden layers in between the input and output layers. Each of the layers may be made up of a plurality of processing nodes that may be referred to as neurons. For example, the automated analysis processor 150 may include an input layer having a neuron for each pixel or a group of pixels from a scan plane of an anatomical structure. The output layer may have a neuron corresponding to a plurality of pre-defined biological and/or artificial structures. As an example, if performing an ultrasound-based regional anesthesia procedure, the output layer may include neurons for a brachial plexus nerve bundle, the axillary artery, beveled regions on anesthetic needles, and the like. Other ultrasound procedures may utilize output layers that include neurons for nerves, vessels, bones, organs, needles, implantable devices, or any suitable biological and/or artificial structure. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the ultrasound image data. The processing performed by the artificial intelligence segmentation processor 140 deep neural network (e.g., convolutional neural network) may identify biological and/or artificial structures in ultrasound image data with a high degree of probability.

For example, the automated analysis processor 150 may include an input layer having a neuron for each pixel or a group of pixels from a scan plan of a biological and/or artificial structure, such as an organ, nerves, vessels, tissue, needle, implantable device, and/or the like. The output layer may have a neuron corresponding to each structure of the biological and/or artificial structure. As an example, if imaging a heart, the output layer may include neurons for a mitral valve, the aortic valve, the tricuspid valve, the pulmonary valve, the left atrium, the right atrium, the left ventricle, the right ventricle, the septum, the papillary muscle, the inferior wall, unknown, and/or other. Other ultrasound procedures may utilize output layers that include neurons for nerves, vessels, bones, organs, needles, implantable devices, or any suitable biological and/or artificial structure. Each neuron of each layer may perform a processing function and pass the processed ultrasound image information to one of a plurality of neurons of a downstream layer for further processing. As an example, neurons of a first layer may learn to recognize edges of structure in the ultrasound image data. The neurons of a second layer may learn to recognize shapes based on the detected edges from the first layer. The neurons of a third layer may learn positions of the recognized shapes relative to landmarks in the volume renderings. The processing performed by the automated analysis processor 150 deep neural network may identify biological and/or artificial structures and the location of the structures in the ultrasound images with a high degree of probability.

The automated analysis processor 150 may comprise suitable logic, circuitry, interfaces and/or code that may be operable to automatically annotate, measure, and/or diagnose the biological and/or artificial structures depicted in the ultrasound image. For example, the automated analysis processor 150 may annotate, measure, and/or diagnose the identified and segmented structures identified by the output layer of the deep neural network. As an example, the automated analysis processor 150 may be utilized to perform measurements of detected anatomical structures. For example, the automated analysis processor 150 may be configured to perform a heart measurement, such as a left ventricle internal diameter at end systole (LVIDs) measurement, an interventricular septum at end systole (IVSs) measurement, a left ventricle posterior wall at end systole (LVPWs) measurement, or an aortic valve diameter (AV Diam) measurement. The annotations, measurements, and/or diagnosis may be overlaid on the ultrasound image and presented at the display system 134 and/or otherwise associated with the ultrasound image. For example, each of the annotations, measurements, and/or diagnosis associated with the ultrasound images may be stored with or in relation to the associated ultrasound image as metadata. In various embodiments, the metadata may include a set of coordinates corresponding with the location of the annotation, measurement, and/or diagnosis in the ultrasound image. The annotations, measurements, and/or diagnosis having the set of coordinates may be stored at archive 138 and/or at any suitable storage medium.

The signal processor 132 may include a data sharing processor 160 that comprises suitable logic, circuitry, interfaces and/or code that may be operable to share the ultrasound images labeled by the labeling processor 140. The data sharing processor 160 may be configured to prompt a user and/or patient to authorize sharing of the labeled images. For example, the data sharing processor 160 may present a prompt at the display system 134 for receiving consent of the user and/or the patient to sharing anonymized data. The labeled images may be uploaded via the communication interface 180 to an automated analysis feature provider, such that the labeled images may be used to train artificial intelligence image analysis algorithms, one or more deep neural networks (e.g., a convolutional neural network) and/or any suitable form of artificial intelligence image analysis techniques or machine learning processing functionality to provide the automated analysis feature(s) and/or tool(s). In various embodiments, the data sharing processor 160 may be configured to capture and share information about the authorizing user at the site, such that the automated analysis feature provider may analyze differences in scanning locations, scanning techniques, image quality, labeling quality, and the like of different authorizing users and/or at different site locations.

The data sharing processor 160 may comprise suitable logic, circuitry, interfaces and/or code that may be operable to anonymize data prior to sharing the data via the communication interface 180. For example, patient identification information, such as names, addresses, and the like may be scrubbed from the labeled image metadata prior to sharing.

The data sharing processor 160 may comprise suitable logic, circuitry, interfaces and/or code that may be operable to enable non-enabled automated analysis features and/or tools. For example, the data sharing processor 160 may enable a specific tool or suite of tools in response to a specified amount of data being shared. In various embodiments, the automated analysis features and/or tools may be provided with tiered levels of access, for example, where the user gains access to a suite of features when a specified level of data is shared. In an exemplary embodiment, the data sharing processor 160 may be configured to provide credits for purchasing or otherwise acquiring non-enabled automated analysis features and/or tools. As an example, a user may receive credits in response to donated data where the credits may be redeemed in an application store accessible via the communication interface 180. For example, the user interface provided at the display system 134 may include links, tabs, or the like for accessing the application store. The application store may provide automated analysis features and/or tools that may be purchased and/or licensed. In various embodiments, the application store may provide a leaderboard listing users in order of amount of data shared to encourage data donation. Additionally and/or alternatively, a data donation leaderboard may be presented within the user interface provided at the display system 134.

Still referring to FIG. 1, the training engine 170 may comprise suitable logic, circuitry, interfaces and/or code that may be operable to train the neurons of the deep neural network(s) of the automated analysis processor 150. For example, the training engine 170 may train the deep neural networks of the automated analysis processor 150 using databases(s) of ultrasound images labeled by the labeling module 140. In various embodiments, the automated analysis processor 150 deep neural network may be trained by the training engine 170 with multiple different viewing angles of ultrasound images having associated structure coordinates to train the automated analysis processor 150 with respect to the characteristics of the particular structure, such as the appearance of structure edges, the appearance of structure shapes based on the edges, the positions of the shapes in the ultrasound image data, and the like. In certain embodiments, the organ may be a heart and the structural information may include information regarding the edges, shapes, positions, and timing information (e.g., end diastole, end systole, etc.) of a mitral valve, aortic valve, pericardium, posterior wall, septal wall, interventricular septum, right ventricle, left ventricle, right atrium, left atrium, and/or the like. In certain embodiments, the training engine 170 and/or training image databases may be external system(s) communicatively coupled via the communication interface 180 to the ultrasound system 100. For example, the training engine 170 and/or training databases may be provided by an automated analysis feature provider. As another example, the automated analysis feature provider may provide the automated analysis processor 150 with the trained artificial intelligence image analysis algorithms, one or more deep neural networks (e.g., a convolutional neural network) and/or any suitable form of artificial intelligence image analysis techniques or machine learning processing functionality to provide the automated analysis feature(s) and/or tool(s).

The display system 134 may be any device capable of communicating visual information to a user. For example, a display system 134 may include a liquid crystal display, a light emitting diode display, and/or any suitable display or displays. The display system 134 can be operable to display information from the signal processor 132 and/or archive 138, such as medical images, labeling tools, automated analysis tools, or any suitable information.

The archive 138 may be one or more computer-readable memories integrated with the ultrasound system 100 and/or communicatively coupled (e.g., over a network) to the ultrasound system 100, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. The archive 138 may include databases, libraries, sets of information, or other storage accessed by and/or incorporated with the signal processor 132, for example. The archive 138 may be able to store data temporarily or permanently, for example. The archive 138 may be capable of storing medical image data, data generated by the signal processor 132, and/or instructions readable by the signal processor 132, among other things. In various embodiments, the archive 138 stores ultrasound images, labeled ultrasound images, ultrasound images processed by the automated analysis processor 150, parameters and settings, and/or instructions for performing labeling, automated analysis, data sharing, and/or training machine learning algorithms, among other things.

The communication interface 180 may comprise suitable logic, circuitry, interfaces and/or code that may be operable to allow communication between the ultrasound system 100 and other external systems, for example. The communication interface 180 may provide wired and/or wireless connections, for example. Wireless connections may include, for example, any combination of short-range, long range, Wi-Fi, cellular, personal communication system (PCS), Bluetooth, Near Field communication (NFC), radio frequency identification (RFID), or any suitable wireless connection. The ultrasound system 100 may singly or as a group with other ultrasound systems and/or medical workstations at a site be connected to a network, such as the Internet, for example, via any suitable combination of wired or wireless data communication links. In various embodiments, selected ultrasound images labeled by a user via the labeling processor 140 may be shared by data sharing processor 160 with an automated feature analysis provider via the communication interface 180.

Components of the ultrasound system 100 may be implemented in software, hardware, firmware, and/or the like. The various components of the ultrasound system 100 may be communicatively linked. Components of the ultrasound system 100 may be implemented separately and/or integrated in various forms. For example, the display system 134 and the user input device 130 may be integrated as a touchscreen display.

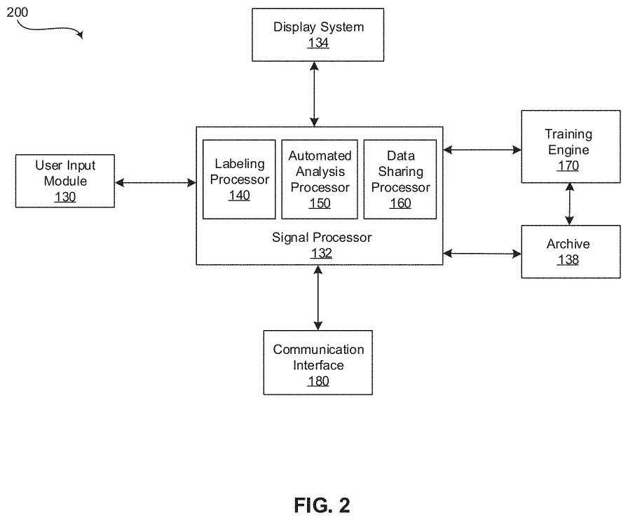

FIG. 2 is a block diagram of an exemplary medical workstation 200 that is operable to prompt data donation for artificial intelligence tool development, in accordance with various embodiments. In various embodiments, components of the medical workstation 200 may share various characteristics with components of the ultrasound system 100, as illustrated in FIG. 1 and described above. Referring to FIG. 2, the medical workstation 200 comprises a display system 134, a signal processor 132, an archive 138, a user input module 130, a training engine 170, and a communication interface 180. Components of the medical workstation 200 may be implemented in software, hardware, firmware, and/or the like. The various components of the medical workstation 200 may be communicatively linked. Components of the medical workstation 200 may be implemented separately and/or integrated in various forms. For example, the display system 134 and the user input module 130 may be integrated as a touchscreen display.

The display system 134 may be any device capable of communicating visual information to a user. As discussed above with respect to FIG. 1, the display system 134 may be operable to display information from the signal processor 132 and/or archive 138, such as medical images, labeling tools, automated analysis tools, or any suitable information.

The signal processor 132 may be one or more central processing units, microprocessors, microcontrollers, and/or the like. The signal processor 132 may be an integrated component, or may be distributed across various locations, for example. The signal processor 132 comprises a labeling processor 140, an automated analysis processor 150, and a data sharing processor 160, as described above with reference to FIG. 1, and may be capable of receiving input information from a user input module 130 and/or archive 138, generating an output displayable by a display system 134, and manipulating the output in response to input information from a user input module 130, among other things. The signal processor 132, labeling processor 140, automated analysis processor 150, and/or data donation processor 160 may be capable of executing any of the method(s) and/or set(s) of instructions discussed herein in accordance with the various embodiments, for example.

The archive 138 may be one or more computer-readable memories integrated with the medical workstation 200 and/or communicatively coupled (e.g., over a network) to the medical workstation 200, such as a Picture Archiving and Communication System (PACS), a server, a hard disk, floppy disk, CD, CD-ROM, DVD, compact storage, flash memory, random access memory, read-only memory, electrically erasable and programmable read-only memory and/or any suitable memory. As described above with respect to FIG. 1, the archive 138 may be configured to store ultrasound images, labeled ultrasound images, ultrasound images processed by the automated analysis processor 150, parameters and settings, and/or instructions for performing labeling, automated analysis, data sharing, and/or training machine learning algorithms, among other things.

The user input module 130 may include any device(s) capable of communicating information from a user and/or at the direction of the user to the signal processor 132 of the medical workstation 200, for example. As discussed above with respect to FIG. 1, the user input module 130 may include a touch panel, button(s), a mousing device, keyboard, rotary encoder, trackball, camera, voice recognition, and/or any other device capable of receiving a user directive.

The training engine 170 may comprise suitable logic, circuitry, interfaces and/or code that may be operable to train the neurons of the deep neural network(s) of the automated analysis processor 150. Additionally and/or alternatively, an automated analysis feature provider may provide the automated analysis processor 150 with the trained artificial intelligence image analysis algorithms, one or more deep neural networks (e.g., a convolutional neural network) and/or any suitable form of artificial intelligence image analysis techniques or machine learning processing functionality to provide the automated analysis feature(s) and/or tool(s).

The communication interface 180 may comprise suitable logic, circuitry, interfaces and/or code that may be operable to allow communication between the ultrasound system 100 and other external systems. As described above with respect to FIG. 1, the communication interface 180 may provide wired and/or wireless connections, for example. In certain embodiments, authorized ultrasound images labeled by a user via the labeling processor 140 may be shared by data sharing processor 160 with an automated feature analysis provider via the communication interface 180.

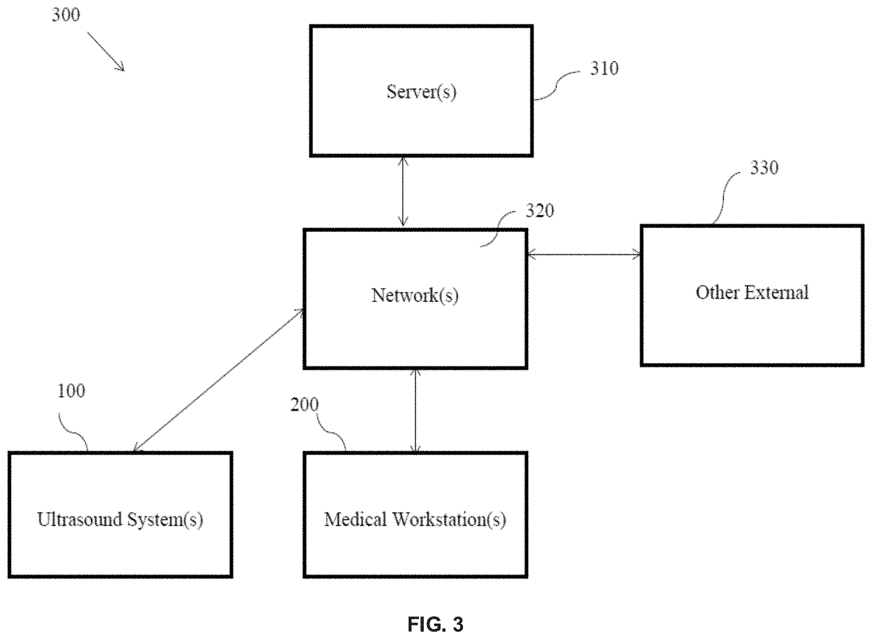

FIG. 3 is a block diagram of an exemplary system 300 in which a representative embodiment may be practiced. As illustrated in FIG. 3, the system 300 includes one or more servers 310. The server(s) 310 may include, for example, web server(s), database server(s), application server(s), and the like. The server(s) 310 may be interconnected, and may singly or as a group be connected to a network 320, such as the Internet, for example, via any suitable combination of wired or wireless data communication links. FIG. 3 also includes external systems 330. The external systems 330 may be interconnected, and may singly or as a group be connected to a network 320, such as the Internet, for example, via any suitable combination of wired or wireless data communication links. The server(s) 310 and/or the external systems 330 may include a signal processor 132 and/or an archive 138 as described above. FIG. 3 includes one or more ultrasound systems 100 and/or medical workstations 200 as described above with reference to FIGS. 1 and 2. The ultrasound systems 100 and/or medical workstations 200 may be connected to the network 320 by any suitable combination of wired or wireless data communication links.

In various embodiments, the server(s) 310 may be operable to automatically annotate, measure, and/or diagnose the biological and/or artificial structures depicted in ultrasound images and/or anonymize and share authorized data. For example, the functionality of one or more of the automated analysis processor 150 and/or data sharing processor 160 may be performed by the server(s) 310 in the background or at the direction of a user via one or both of the ultrasound systems 100 and/or the medical workstations 200. The ultrasound image data processed and stored by the server(s) 310 may be accessed by the ultrasound systems 100 and/or the medical workstations 200 via network(s) 320.

In certain embodiments, the external systems 330 may be an automated analysis feature provider operable to provide the automated analysis processor 150 with the trained artificial intelligence image analysis algorithms, one or more deep neural networks (e.g., a convolutional neural network) and/or any suitable form of artificial intelligence image analysis techniques or machine learning processing functionality to provide the automated analysis feature(s) and/or tool(s). For example, the functionality of training engine 170 described above with respect to FIGS. 1 and 2 may be performed by the external systems 330 and provided to one or both of the ultrasound systems 100 and/or the medical workstations 200. The automated analysis feature(s) and/or tool(s) generated and stored by the external systems 330 may be accessed by the ultrasound systems 100 and/or the medical workstations 200 via network(s) 320.

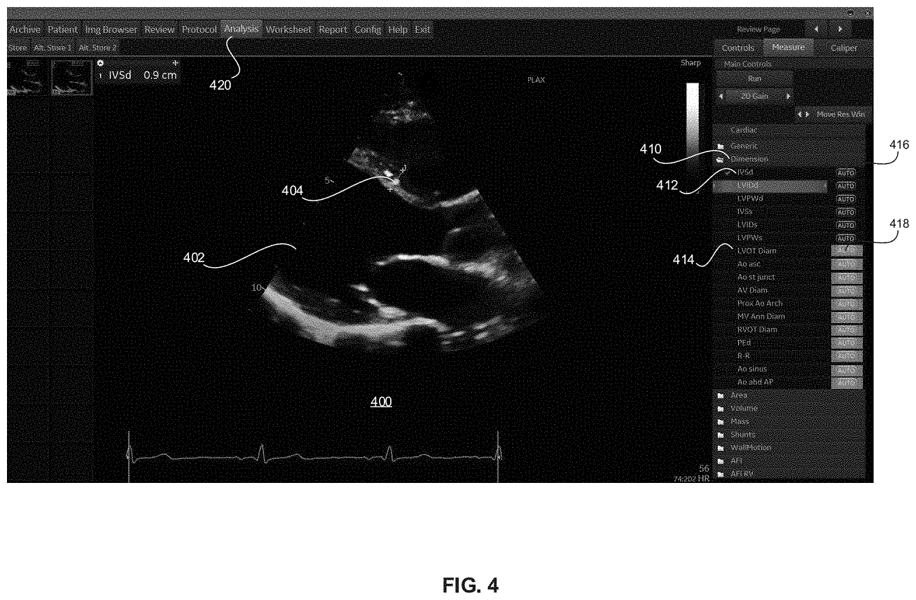

FIG. 4 is a display 400 of an exemplary ultrasound image 402 and tools 412, 414 for analyzing the ultrasound image, in accordance with various embodiments. Referring to FIG. 4, the display 400 comprises an ultrasound image 402, automated analysis feature categories 410 having automated analysis features 412, 414, and user interface tabs 420. The ultrasound image 402 may be overlaid and/or otherwise associated with annotations, measurements 404, diagnosis, and the like. For example, the ultrasound image 402 illustrated in FIG. 4 includes a measurement 404 of an interventricular septum at end diastole (IVSd). The annotations, measurements, diagnosis, and the like may be provided manually using labeling tools and/or automatically using automated analysis features 412, 414. In various embodiments, the automated analysis features 412, 414 may be grouped and/or otherwise organized in categories 410 and/or sub-categories. For example, the automated analysis features 412, 414 may be provided for different measurement types and for different applications. As an example, a cardiac application may include categories for generic measurements, dimension measurements 410, area measurements, volume measurements, mass measurements, and the like. Each of the categories may include specific measurements for that category. For example, the dimension measurements category 410 may include a left ventricle internal diameter at end diastole (LVIDd) measurement, an interventricular septum at end diastole (IVSd) measurement 412, and a left ventricle posterior wall at end diastole (LVPWd) measurement, among other things. In a representative embodiment, one or more of the automated analysis features 412 may be non-enabled 416 and/or one or more of the automated analysis features 414 may be enabled 418. The display 400 of the automated analysis features 412, 414 may include markers, shading 416, highlighting 418, and/or any suitable identifier for specifying whether the feature 412, 414 is non-enabled 416 or enabled 418, for example. The user interface tabs 420 may allow a user to navigate the user interface to the desired functionality. In various embodiments, the user interface tabs 420 may include a tab for accessing image analysis functionality, a tab to access an application store to purchase access to automated analysis features 412, 414, and/or any suitable tab functionality.

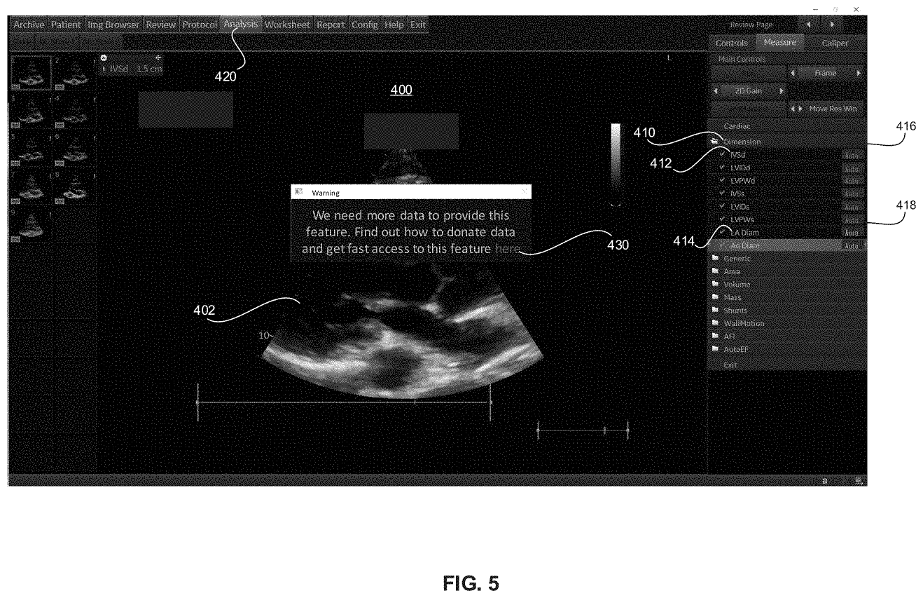

FIG. 5 is a display 400 of an exemplary ultrasound image 402, tools for analyzing the ultrasound image 412, 414, and a prompt for donating data 430, in accordance with various embodiments. The features provided in the display 400 of FIG. 5 may share various characteristics with the features provided in the display 400 of FIG. 4 as described above. Referring to FIG. 5, the display 400 comprises an ultrasound image 402, automated analysis feature categories 410 having automated analysis features 412, 414, user interface tabs 420, and a data donation prompt 430. In various embodiments, the data donation prompt 430 may be presented upon a user selection of a non-enabled 416 automated analysis feature 412. For example, with reference to FIG. 5, if a user selects an interventricular septum at end diastole (IVSd) measurement 412, which is non-enabled as shown by the grayed out “Auto” box 416, a prompt 430 may be presented to allow a user to share anonymized labeled data that may be used to develop artificial intelligence tools, such as the automated analysis features 412, 414. In various embodiments, the non-enabled automated analysis feature 412 may be enabled if one or more conditions are met. For example, the conditions for enabling one or more automated analysis features 412, 414 may include agreeing to donate data, donating a pre-determined amount of data, and the like. In various embodiments, a user may be awarded credits based on an amount of donated data for enabling one or more automated analysis features 412, 414, such as via an application store.

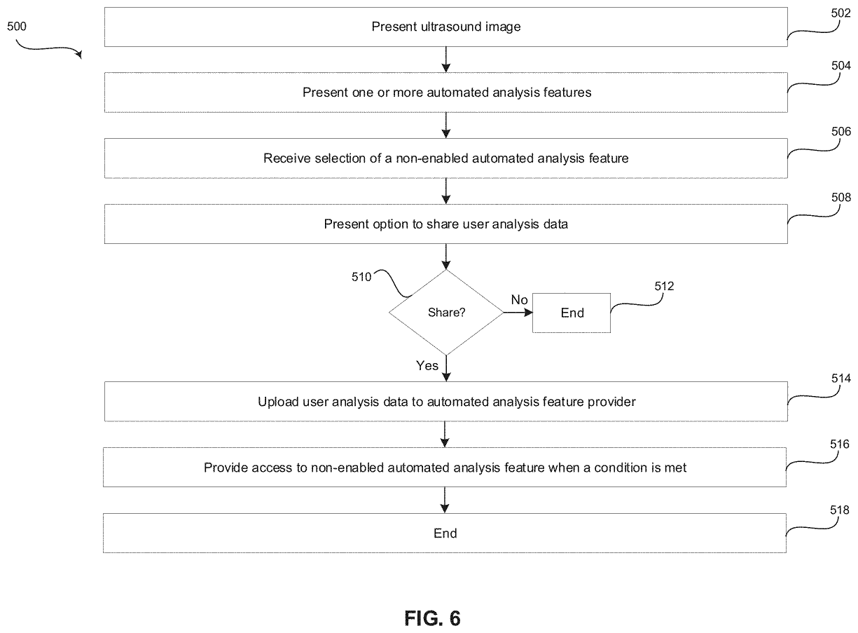

FIG. 6 is a flow chart 500 illustrating exemplary steps 502-518 that may be utilized for prompting data donation for artificial intelligence tool development, in accordance with various embodiments. Referring to FIG. 6, there is shown a flow chart 500 comprising exemplary steps 502 through 518. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

At step 502, an ultrasound system 100 or medical workstation 200 presents an ultrasound image 402. For example, the ultrasound system 100 may acquire an ultrasound image 402 with an ultrasound probe 104 positioned at a scan position over region of interest and may present the ultrasound image 402 at a display system 134. As another example, the ultrasound system 100 or medical workstation 200 may retrieve the ultrasound image 402 from archive 138 or any suitable data storage medium and present the image 402 at the display system 134.

At step 504, the ultrasound system 100 or medical workstation 200 presents one or more automated analysis features 412, 414 For example, an automated analysis processor 150 of a signal processor 132 may be configured to present automated analysis features 412, 414 with the ultrasound image 402 presented at step 502. In various embodiments, the automated analysis features 412, 414 may include tools for automatically annotating, measuring, and/or providing diagnosis to the ultrasound image 402. In certain embodiments, the automated analysis features 412, 414 may include enabled 414 and/or non-enabled tools 412. For example, the automated analysis features 412, 414 may be presented with an identifier 416, 418 designating whether the tool is enabled or non-enabled.

At step 506, a signal processor 132 of the ultrasound system 100 or medical workstation 200 may receive a selection of a non-enabled automated analysis feature 412. For example, the automated analysis processor 150 and/or a data sharing processor 160 of the signal processor 132 may receive a user selection of a non-enabled automated analysis feature 412 via a user input device 130.

At step 508, the signal processor 132 of the ultrasound system 100 or medical workstation 200 may present an option 430 to share user analysis data. For example, the data sharing processor 160 of the signal processor 132 may be configured to present a prompt 430 at a display 400 of the display system 134. The prompt 430 may provide options or a link to options for authorizing data sharing. The prompt 430 may request the consent of the user and/or patient to share analysis data. The analysis data may include manually labeled images and information regarding the user at the site. For example, the analysis data may include anonymized images having annotations, measurements, and/or diagnosis. As another example, the analysis data may include information regarding the medical personnel performing the analysis.

At step 510, the signal processor 132 of the ultrasound system 100 or medical workstation 200 receives a user instruction to share the analysis data or a user instruction not to share the analysis data. For example, the data sharing processor 160 of the signal processor 132 may receive an instruction not to share the analysis data and the process 500 then ends at step 512. As another example, the data sharing processor 160 of the signal processor 132 may receive an instruction authorizing the donation of the analysis data and the process proceeds to step 514.

At step 514, the signal processor 132 of the ultrasound system 100 or medical workstation 200 uploads user analysis data to an automated analysis feature provider. For example, the data sharing processor 160 of the signal processor 132 select analysis data that the user and/or patient has authorized to share, anonymize the analysis data to remove personal patient identifying information, and transmits the anonymized analysis data to an automated analysis feature provider via a communication interface 180. The user analysis data may include ultrasound images having annotations, measurements, and/or diagnosis provided by the medical personnel user. In various embodiments, the user analysis data may include information regarding the medial personnel performing the analysis being donated. The shared analysis data may be used by the automated analysis feature provider to develop artificial intelligence tools 412, 414.

At step 516, the signal processor 132 of the ultrasound system 100 or medical workstation 200 provides access to the non-enabled automated analysis feature 412 when a condition is met. For example, the data sharing processor 160 of the signal processor 132 may enable a selected non-enabled automated analysis feature 412 when the condition is met. The condition may include one or more of the authorization to share the user analysis data, a specified amount of user analysis data that is shared, and/or any suitable condition. In various embodiments, the data sharing processor 160 may provide tiered levels of access where a user may gain access to a suite of features when a specified level of data is shared. In certain embodiments, the data sharing processor 160 may provide credits corresponding to the amount of user analysis data that is shared. The credits may be used to purchase access to one or more non-enabled automated analysis features 412. For example, the credits may be applied at the user interface display 400 and/or via an application store. The application store may be provided as part of the user interface display 400 and/or may be linked through the user interface display 400, among other things. The process 500 ends at step 518 when the selected automated analysis feature is enabled by the data sharing processor 160.

Aspects of the present disclosure provide methods 500 and systems 100, 200, 300 for prompting data donation for artificial intelligence tool development. In accordance with various embodiments, the method 500 may comprise presenting 502, 504, by a system 100, 200, 300, an ultrasound image 402 and at least one automated analysis feature 412, 414 at a display system 134 of the system 100, 200, 300. The at least one automated analysis feature 412, 414 comprises one or more non-enabled automated analysis features 412. The method 500 may comprise receiving 506, by at least one processor 134, 150, 160 of the system 100, 200, 300, a user selection of at least one of the one or more non-enabled automated analysis features 412. The method 500 may comprise presenting 508 at the display system 134, by the at least one processor 132, 150, 160, a prompt 430 providing a user option to share user analysis data. The method 500 may comprise receiving 510, by the at least one processor 132, 150, 160, a user selection opting to share the user analysis data. The method 500 may comprise providing access 516, by the at least one processor 132, 150, 160, to the at least one of the one or more non-enabled automated analysis features 412 when at least one condition is met.

In a representative embodiment, the system 100, 200, 300 may be a medical workstation 200 or an ultrasound system 100. In an exemplary embodiment, the at least one condition may comprise one or both of the user selection opting to share the user analysis data, and sharing a specified amount of the user analysis data. In various embodiments, the user analysis data may comprise ultrasound images 402 labeled with at least one annotation, at least one measurement 404, and/or at least one diagnosis. In certain embodiments, the user analysis data may further comprise information about a user of the system. In a representative embodiment, the method 500 may comprise in response to receiving the user selection opting to share the user analysis data 510, anonymizing, by the at least one processor 132, 140, 160, the user analysis data and sharing 514, by the at least one processor 132, 140, 160, the user analysis data. In an exemplary embodiment, the method 500 may comprise presenting 508, by the at least one processor 132, 150, 160, a patient prompt 430 requesting patient consent to share the user analysis data. In certain embodiments, the at least one of the one or more non-enabled automated analysis features 412 may be a suite of non-enabled automated analysis features and access is provided, by the at least one processor 132, 150, 160, to the suite of non-enabled automated analysis features 412 when a specified level of data is shared.

Various embodiments provide a system 100, 200, 300 for prompting data donation for artificial intelligence tool development. The system 100, 200, 300 may comprise a display system 134 and at least one processor 132, 140, 150, 160. The display system 134 may be configured to present an ultrasound image 402 and at least one automated analysis feature 412, 414. The at least one automated analysis feature 412, 414 may comprise one or more non-enabled automated analysis features 412. The at least one processor 132, 150, 160 may be configured to receive a user selection of at least one of the one or more non-enabled automated analysis features 412. The at least one processor 132, 150, 160 may be configured to present, at the display system 134, a prompt 430 providing a user option to share user analysis data. The at least one processor 132, 150, 160 may be configured to receive a user selection opting to share the user analysis data. The at least one processor 132, 150, 160 may be configured to provide access to the at least one of the one or more non-enabled automated analysis features 412 when at least one condition is met.

In an exemplary embodiment, the system 100, 200, 300 may be a medical workstation 200 or an ultrasound system 100. In various embodiments, the at least one condition may comprise one or both of the user selection opting to share the user analysis data, and sharing a specified amount of the user analysis data. In certain embodiments, the user analysis data may comprise ultrasound images 402 labeled with at least one annotation, at least one measurement 404, and/or at least one diagnosis and information about a user of the system 100, 200, 300. In a representative embodiment, in response to receiving the user selection opting to share the user analysis data, the at least one processor 132, 140, 160 may be configured to anonymize the user analysis data and share the user analysis data. In an exemplary embodiment, the at least one processor 132, 150, 160 may be configured to present a patient prompt 430 requesting patient consent to share the user analysis data. In various embodiments, the at least one of the one or more non-enabled automated analysis features 412 is a suite of non-enabled automated analysis features and the at least one processor 132, 150, 160 is configured to provide access to the suite of non-enabled automated analysis features 412 when a specified level of data is shared.

Certain embodiments provide a non-transitory computer readable medium having stored thereon, a computer program having at least one code section. The at least one code section is executable by a machine for causing the machine to perform steps 500. The steps 500 may comprise presenting 502, 504 an ultrasound image 402 and at least one automated analysis feature 412, 414 at a display system 134. The at least one automated analysis feature 412, 414 may comprise one or more non-enabled automated analysis features 412. The steps 500 may comprise receiving 506 a user selection of at least one of the one or more non-enabled automated analysis features 412. The steps 500 may comprise presenting 508 at the display system 134 a prompt 430 providing a user option to share user analysis data. The steps 500 may comprise receiving 510 a user selection opting to share the user analysis data. The steps 500 may comprise providing 516 access to the at least one of the one or more non-enabled automated analysis features 412 when at least one condition is met.

In various embodiment, the at least one condition may comprise one or both of the user selection opting to share the user analysis data and sharing a specified amount of the user analysis data. In certain embodiments, the user analysis data may comprise ultrasound images 402 labeled with at least one annotation, at least one measurement 404, and/or at least one diagnosis and information about a user of a system 100, 200, 300. In a representative embodiment, the steps 500 may comprise in response to receiving the user selection opting to share the user analysis data 510, anonymizing 514 the user analysis data and sharing 514 the user analysis data. In an exemplary embodiment, the at least one of the one or more non-enabled automated analysis features 412 may be a suite of non-enabled automated analysis features, and access is provided to the suite of non-enabled automated analysis features 412 when a specified level of data is shared.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” and/or “configured” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for prompting data donation for artificial intelligence tool development.

Accordingly, the present disclosure may be realized in hardware, software, or a combination of hardware and software. The present disclosure may be realized in a centralized fashion in at least one computer system, or in a distributed fashion where different elements are spread across several interconnected computer systems. Any kind of computer system or other apparatus adapted for carrying out the methods described herein is suited.

Various embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method comprising:
presenting, by a system, an ultrasound image and at least one automated analysis feature at a display system of the system, wherein the at least one automated analysis feature comprises one or more non-enabled automated analysis features;
receiving, by at least one processor of the system, a user selection of at least one of the one or more non-enabled automated analysis features;
presenting at the display system, by the at least one processor, a prompt providing a user option to share user analysis data;
receiving, by the at least one processor, a user selection opting to share the user analysis data; and
providing access, by the at least one processor, to the at least one of the one or more non-enabled automated analysis features when at least one condition is met.

2. The method of claim 1, wherein the system is a medical workstation or an ultrasound system.

3. The method of claim 1, wherein the at least one condition comprises one or both of:
the user selection opting to share the user analysis data, and
sharing a specified amount of the user analysis data.

4. The method of claim 1, wherein the user analysis data comprises ultrasound images labeled with at least one annotation, at least one measurement, and/or at least one diagnosis.

5. The method of claim 4, wherein the user analysis data further comprises information about a user of the system.

6. The method of claim 1, comprising:
in response to receiving the user selection opting to share the user analysis data:
anonymizing, by the at least one processor, the user analysis data; and
sharing, by the at least one processor, the user analysis data.

7. The method of claim 1, comprising presenting, by the at least one processor, a patient prompt requesting patient consent to share the user analysis data.

8. The method of claim 1, wherein:
the at least one of the one or more non-enabled automated analysis features is a suite of non-enabled automated analysis features; and
access is provided, by the at least one processor, to the suite of non-enabled automated analysis features when a specified level of data is shared.

9. A system comprising:
a display system configured to present an ultrasound image and at least one automated analysis feature, wherein the at least one automated analysis feature comprises one or more non-enabled automated analysis features;
at least one processor configured to:
receive a user selection of at least one of the one or more non-enabled automated analysis features;
present, at the display system, a prompt providing a user option to share user analysis data;
receive a user selection opting to share the user analysis data; and
provide access to the at least one of the one or more non-enabled automated analysis features when at least one condition is met.

10. The system of claim 9, wherein the system is a medical workstation or an ultrasound system.

11. The system of claim 9, wherein the at least one condition comprises one or both of:
the user selection opting to share the user analysis data, and
sharing a specified amount of the user analysis data.

12. The system of claim 9, wherein the user analysis data comprises:
ultrasound images labeled with at least one annotation, at least one measurement, and/or at least one diagnosis; and
information about a user of the system.

13. The system of claim 9, wherein in response to receiving the user selection opting to share the user analysis data, the at least one processor is configured to:
anonymize the user analysis data; and
share the user analysis data.

14. The system of claim 9, wherein the at least one processor is configured to present a patient prompt requesting patient consent to share the user analysis data.

15. The system of claim 9, wherein:
the at least one of the one or more non-enabled automated analysis features is a suite of non-enabled automated analysis features; and
the at least one processor is configured to provide access to the suite of non-enabled automated analysis features when a specified level of data is shared.

16. A non-transitory computer readable medium having stored thereon, a computer program having at least one code section, the at least one code section being executable by a machine for causing the machine to perform steps comprising:
presenting an ultrasound image and at least one automated analysis feature at a display system, wherein the at least one automated analysis feature comprises one or more non-enabled automated analysis features;
receiving a user selection of at least one of the one or more non-enabled automated analysis features;
presenting at the display system a prompt providing a user option to share user analysis data;
receiving a user selection opting to share the user analysis data; and
providing access to the at least one of the one or more non-enabled automated analysis features when at least one condition is met.

17. The non-transitory computer readable medium of claim 16, wherein the at least one condition comprises one or both of:
the user selection opting to share the user analysis data, and
sharing a specified amount of the user analysis data.

18. The non-transitory computer readable medium of claim 16, wherein the user analysis data comprises:
ultrasound images labeled with at least one annotation, at least one measurement, and/or at least one diagnosis; and
information about a user of a system.

19. The non-transitory computer readable medium of claim 16, comprising:
in response to receiving the user selection opting to share the user analysis data:
anonymizing the user analysis data; and
sharing the user analysis data.

20. The non-transitory computer readable medium of claim 16, wherein:
the at least one of the one or more non-enabled automated analysis features is a suite of non-enabled automated analysis features; and
access is provided to the suite of non-enabled automated analysis features when a specified level of data is shared.
